(19) United States
(12) Patent Application Publication
(10) Pub. No.: US 2013/0005742 A1
(43) Pub. Date: Jan. 3, 2013

(54) Substituted Diphenylpyrazine Derivatives

(76) Inventors: Scott L. Harbeson, Cambridge, MA (US); Craig E. Masse, Cambridge, MA (US); Julie F. Liu, Lexington, MA (US)

(21) Appl. No.: 13/388,806

(22) PCT Filed: Aug. 6, 2010
(86) PCT No.: PCT/US10/44704

§ 371 Date: Sep. 17, 2012

Related U.S. Application Data
(60) Provisional application No. 61/231,878, filed on Aug. 6, 2009.

(51) Int. Cl. A61K 31/4965; A61P 7/02; A61P 9/00; A61P 9/10; C07D 241/20; A61P 9/12

(52) U.S. Cl. 514/255.06; 544/336

(57) ABSTRACT

This invention relates to novel substituted diphenylpyrazines and pharmaceutically acceptable salts thereof. This invention also provides compositions comprising a compound of this invention and the use of such compositions in methods of treating diseases and conditions that are beneficially treated by administering a PGI₂ receptor agonist.

### RELATED APPLICATION

This application claims the benefit of U.S. Provisional Application No. 61/231,878 filed on Aug. 6, 2009, the entire teachings of which are incorporated herein by reference.

### BACKGROUND OF THE INVENTION

Many current medicines suffer from poor absorption, distribution, metabolism and/or excretion (ADME) properties that prevent their wider use or limit their use in certain indications. Poor ADME properties are also a major reason for the failure of drug candidates in clinical trials. While formulation technologies and prodrug strategies can be employed in some cases to improve certain ADME properties, these approaches often fail to address the underlying ADME problems that exist for many drugs and drug candidates. One such problem is rapid metabolism that causes a number of drugs, which otherwise would be highly effective in treating a disease, to be cleared too rapidly from the body. A possible solution to rapid drug clearance is frequent or high dosing to attain a sufficiently high plasma level of drug. This, however, introduces a number of potential treatment problems such as poor patient compliance with the dosing regimen, side effects that become more acute with higher doses, and increased cost of treatment. A rapidly metabolized drug may also expose patients to undesirable toxic or reactive metabolites.

Another ADME limitation that affects many medicines is the formation of toxic or biologically reactive metabolites. As a result, some patients receiving the drug may experience toxicities, or the safe dosing of such drugs may be limited such that patients receive a suboptimal amount of the active agent. In certain cases, modifying dosing intervals or formulation approaches can help to reduce clinical adverse effects, but often the formation of such undesirable metabolites is intrinsic to the metabolism of the compound.

In some select cases, a metabolic inhibitor will be co-administered with a drug that is cleared too rapidly. Such is the case with the protease inhibitor class of drugs that are used to treat HIV infection. The FDA recommends that these drugs be co-dosed with ritonavir, an inhibitor of cytochrome P450 enzyme 3A4 (CYP3A4), the enzyme typically responsible for their metabolism (see Kempf, D. J. et al., Antimicrobial agents and chemotherapy, 1997, 41(3): 654-60). Ritonavir, however, causes adverse effects and adds to the pill burden for HIV patients who must already take a combination of different drugs. Similarly, the CYP2D6 inhibitor quinidine has been added to dextromethorphan for the purpose of reducing rapid CYP2D6 metabolism of dextromethorphan in a treatment of pseudobulbar affect. Quinidine, however, has unwanted side effects that greatly limit its use in potential combination therapy (see Wang, L et al., Clinical Pharmacology and Therapeutics, 1994, 56(6 Pt 1): 659-67; and FDA label for quinidine at www.accessdata.fda.gov).

In general, combining drugs with cytochrome P450 inhibitors is not a satisfactory strategy for decreasing drug clearance. The inhibition of a CYP enzyme's activity can affect the metabolism and clearance of other drugs metabolized by that same enzyme. CYP inhibition can cause other drugs to accumulate in the body to toxic levels.

A potentially attractive strategy for improving a drug's metabolic properties is deuterium modification. In this approach, one attempts to slow the CYP-mediated metabolism of a drug or to reduce the formation of undesirable metabolites by replacing one or more hydrogen atoms with deuterium atoms. Deuterium is a safe, stable, non-radioactive isotope of hydrogen. Compared to hydrogen, deuterium forms stronger bonds with carbon. In select cases, the increased bond strength imparted by deuterium can positively impact the ADME properties of a drug, creating the potential for improved drug efficacy, safety, and/or tolerability. At the same time, because the size and shape of deuterium are essentially identical to those of hydrogen, replacement of hydrogen by deuterium would not be expected to affect the biochemical potency and selectivity of the drug as compared to the original chemical entity that contains only hydrogen.

Over the past 35 years, the effects of deuterium substitution on the rate of metabolism have been reported for a very small percentage of approved drugs (see, e.g., Blake, M I et al, J Pharm Sci, 1975, 64:367-91; Foster, A B, Adv Drug Res 1985, 14:1-40 (“Foster”); Kushner, D J et al, Can J Physiol Pharmacol 1999, 79-88; Fisher, M B et al, Curr Opin Drug Discov Devel, 2006, 9:101-09 (“Fisher”)). The results have been variable and unpredictable. For some compounds deuteration caused decreased metabolic clearance in vivo. For others, there was no change in metabolism. Still others demonstrated increased metabolic clearance. The variability in deuterium effects has also led experts to question or dismiss deuterium modification as a viable drug design strategy for inhibiting adverse metabolism (see Foster at p. 35 and Fisher at p. 101).

The effects of deuterium modification on a drug's metabolic properties are not predictable even when deuterium atoms are incorporated at known sites of metabolism. Only by actually preparing and testing a deuterated drug can one determine if and how the rate of metabolism will differ from that of its non-deuterated counterpart. See, for example, Fukuto et al. (J. Med. Chem. 1991, 34, 2871-76). Many drugs have multiple sites where metabolism is possible. The site(s) where deuterium substitution is required and the extent of deuteration necessary to see an effect on metabolism, if any, will be different for each drug.

NS-304, also known as MRE-304, ACT-293987, 2-[4-[N-(5,6-diphenylpyrazin-2-yl)-N-isopropylamino]butoxy]-N-(methylsulfonyl)acetamide and as N-[2-[4-[N-(5,6-diphenylpyrazin-2-yl)-N-isopropylamino]butoxy]acetyl]methanesulfonamide, is a prodrug of MRE-269. MRE-269 and, to a lesser extent, NS-304 act as PGI₂ receptor agonists. NS-304 is undergoing clinical studies for a variety of clinical indications, including various vascular diseases such as pulmonary arterial hypertension and chronic thromboembolic pulmonary hypertension. PGI₂ receptor agonists may be useful in the treatment of diseases that may be treated by inhibition of platelet aggregation, vasodilation, inhibition of lipid deposition, and/or inhibition of leukocyte activation. Such diseases include pulmonary arterial hypertension, peripheral vascular diseases (for example, arteriosclerosis obliterans, intermittent claudication, peripheral arterial embolism, vibration disease, and Raynaud's disease), systemic lupus erythematosus, reocclusion or restenosis after percutaneous transluminal coronary angioplasty (PTCA), arteriosclerosis, thrombosis, diabetic neuropathy, diabetic nephropathy, hypertension, ischemic diseases (for example, cerebral infarction and myocardial infarction), transient ischemic attack and glomerulonephritis, or acceleration of angiogenesis in peripheral blood vessel reconstruction technique or angiogenesis therapy.

This invention relates to novel substituted derivatives of NS-304 and MRE-269 that have improved properties over NS-304 or MRE-269.

### DEFINITIONS

The term “treat” means decrease, suppress, attenuate, diminish, arrest, or stabilize the development or progression of a disease (e.g., a disease or disorder delineated herein)), lessen the severity of the disease or improve the symptoms associated with the disease.

Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of NS-304 or MRE-269 will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada, E et al., Seikagaku, 1994, 66:15; Gannes, L Z et al., Comp Biochem Physiol Mol Integr Physiol, 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

The term “isotopologue” refers to a species in which the chemical structure differs from a specific compound of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The pharmaceutically acceptable salt may also be a salt of a compound of the present invention having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

The compounds of the present invention (e.g., compounds of Formula I), may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free of another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure without specifying the stereochemistry and has one or more chiral centers, it is understood to represent all possible stereoisomers of the compound or mixtures thereof.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” and “d” both refer to deuterium. “Stereoisomer” refers to both enantiomers and diastereomers. “Tert” and “t-” each refer to tertiary. “US” refers to the United States of America.

Throughout this specification, a variable may be referred to generally (e.g.,“each R”) or may be referred to specifically (e.g., R¹, R², R³, etc.). Unless otherwise indicated, when a variable is referred to generally, it is meant to include all specific embodiments of that particular variable.

### THERAPEUTIC COMPOUNDS

The present invention provides a compound of Formula I:

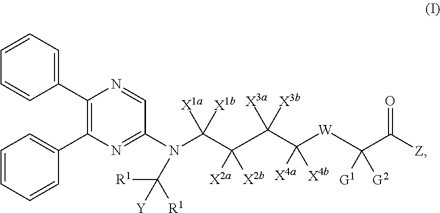

or a pharmaceutically acceptable salt thereof, wherein:

W is O, CH₂ or CD₂;

each of G¹ and G² is independently hydrogen or deuterium;

each R¹ is independently —CD₃, —CD₂H, —CDH₂, or —CH₃;

Z is —OH, —NHSO₂CH₃ or —NHSO₂CD₃;

each of X^{1a}, X^{1b}, X^{2a}, X^{2b}, X^{3a}, X^{3b}, X^{4a} and X^{4b} is independently selected from hydrogen and deuterium; and

Y is independently selected from hydrogen and deuterium; provided that if each R¹ is —CH₃ and each of X^{1a}, X^{1b}, X^{2a}, X^{2b}, X^{3a}, X^{3b}, X^{4a} and X^{4b} is hydrogen, then Y is deuterium. Exemplary values for the variables in Formula (I) are provided in the following paragraphs.

In one embodiment, each R¹ is independently —CD₃ or —CH₃. In one aspect, each R¹ is —CD₃. In another aspect, each R¹ is —CH₃. In one aspect, Y is hydrogen. In another aspect, Y is deuterium.

In one embodiment, Z is —OH. In another embodiment, Z is —NHSO₂CH₃ or —NHSO₂CD₃.

In one embodiment, Y is hydrogen. In another embodiment, Y is deuterium.

In one embodiment, X^{1a}═X^{1b}; X^{2a}═X^{2b}; X^{3a}═X^{3b}; and/or X^{4a}═X^{4b}.

In one embodiment, X^{1a}═X^{1b}. In one aspect of this embodiment, X^{1a}═X^{1b}=deuterium. In another aspect of this embodiment, X^{1a}═X^{1b}=hydrogen.

In one embodiment, X^{2a}═X^{2b}. In one aspect of this embodiment, X^{2a}═X^{2b}=deuterium. In another aspect of this embodiment, X^{2a}═X^{2b}=hydrogen.

In one embodiment, X^{3a}═X^{3b}. In one aspect of this embodiment, X^{3a}═X^{3b}=deuterium. In another aspect of this embodiment, X^{3a}═X^{3b}=hydrogen.

In one embodiment, X^{4a}═X^{4b}. In one aspect of this embodiment, X^{4a}═X^{4b}=deuterium. In another aspect of this embodiment, X^{4a}═X^{4b}=hydrogen.

In one embodiment, each R¹ is —CD₃ or —CH₃; X^{1a}═X^{1b}; X^{2a}═X^{2b}; X^{3a}═X^{3b}; and X^{4a}═X^{4b}.

In one embodiment, every X is deuterium. In one embodiment, every X is hydrogen.

In one embodiment, X^{2a}═X^{2b}═X^{3a}═X^{3b}=deuterium.

In one embodiment, X^{1a}═X^{1b}═X^{4a}═X^{4b}=deuterium.

In one embodiment, X^{1a}═X^{1b}═X^{2a}═X^{2b}=deuterium.

In one embodiment, X^{3a}═X^{3b}═X^{4a}═X^{4b}=deuterium.

In one embodiment, X^{1a}═X^{1b}═X^{3a}═X^{3b}=deuterium.

In one embodiment, X^{2a}═X^{2b}═X^{4a}═X^{4b}=deuterium.

In one embodiment, X^{1a}═X^{1b}=deuterium.

In one embodiment, X^{4a}═X^{4b}=deuterium.

In one embodiment, W is CH₂.

In one embodiment, W is CD₂.

In one embodiment, each of G¹ and G² is deuterium.

In one embodiment, the compound of Formula I is a compound of Formula (Ia):

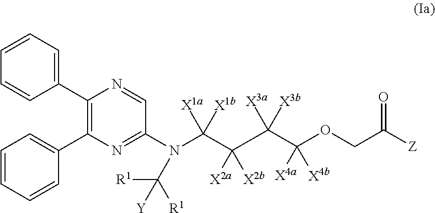

or a pharmaceutically acceptable salt thereof, wherein each X, each R¹, Y and Z are as defined for Formula I.

Exemplary values for the variables in Formula Ia are as provided for Formula (I) and as provided below. In one embodiment, the compound of Formula Ia is a compound wherein Z is —OH and X^{1a,} X^{1b}, X^{2a}, X^{2b}, X^{3a}, X^{3b}, X^{4a}, X^{4b}, R¹ and Y are as shown in Table 1:

TABLE 1
Examples of Compounds of Formula Ia wherein Z is —OH.

| Com- | | | | | | | | | | |
|---|---|---|---|---|---|---|---|---|---|---|
| pound | X^{1a} | X^{1b} | X^{2a} | X^{2b} | X^{3a} | X^{3b} | X^{4a} | X^{4b} | R¹ | Y |
| 100 | D | D | D | D | D | D | D | D | CD₃ | D |
| 101 | D | D | D | D | D | D | D | D | CD₃ | H |
| 102 | D | D | D | D | D | D | D | D | CH₃ | D |
| 103 | D | D | D | D | D | D | D | D | CH₃ | H |
| 104 | H | H | D | D | D | D | H | H | CD₃ | D |
| 105 | H | H | D | D | D | D | H | H | CD₃ | H |
| 106 | H | H | D | D | D | D | H | H | CH₃ | D |
| 107 | H | H | D | D | D | D | H | H | CH₃ | H |
| 108 | D | D | H | H | H | H | D | D | CD₃ | D |
| 109 | D | D | H | H | H | H | D | D | CD₃ | H |
| 110 | D | D | H | H | H | H | D | D | CH₃ | D |
| 111 | D | D | H | H | H | H | D | D | CH₃ | H |
| 112 | D | D | D | D | H | H | H | H | CD₃ | D |
| 113 | D | D | D | D | H | H | H | H | CD₃ | H |
| 114 | D | D | D | D | H | H | H | H | CH₃ | D |
| 115 | D | D | D | D | H | H | H | H | CH₃ | H |
| 116 | H | H | H | H | D | D | D | D | CD₃ | D |
| 117 | H | H | H | H | D | D | D | D | CD₃ | H |
| 118 | H | H | H | H | D | D | D | D | CH₃ | D |
| 119 | H | H | H | H | D | D | D | D | CH₃ | H |
| 120 | D | D | H | H | D | D | H | H | CD₃ | D |
| 121 | D | D | H | H | D | D | H | H | CD₃ | H |
| 122 | D | D | H | H | D | D | H | H | CH₃ | D |
| 123 | D | D | H | H | D | D | H | H | CH₃ | H |
| 124 | H | H | D | D | H | H | D | D | CD₃ | D |
| 125 | H | H | D | D | H | H | D | D | CD₃ | H |
| 126 | H | H | D | D | H | H | D | D | CH₃ | D |
| 127 | H | H | D | D | H | H | D | D | CH₃ | H |
| 128 | D | D | H | H | H | H | H | H | CD₃ | D |
| 129 | D | D | H | H | H | H | H | H | CD₃ | H |
| 130 | D | D | H | H | H | H | H | H | CH₃ | D |
| 131 | D | D | H | H | H | H | H | H | CH₃ | H |
| 132 | H | H | H | H | H | H | D | D | CD₃ | D |
| 133 | H | H | H | H | H | H | D | D | CD₃ | H |
| 134 | H | H | H | H | H | H | D | D | CH₃ | D |
| 135 | H | H | H | H | H | H | D | D | CH₃ | H |
| 136 | H | H | H | H | H | H | H | H | CD₃ | D |
| 137 | H | H | H | H | H | H | H | H | CD₃ | H |
| 138 | H | H | H | H | H | H | H | H | CH₃ | D |

or a pharmaceutically acceptable salt of any of the foregoing compounds.

In another embodiment, the compound of Formula Ia is a compound wherein Z is —NHSO₂CH₃ and X^{1a,} X^{1b}, X^{2a}, X^{2b}, X^{3a}, X^{3b}, X^{4a}, X^{4b}, R¹ and Y are as shown in Table 2:

TABLE 2
Examples of Compounds of Formula Ia
wherein Z is —NHSO₂CH₃.

| Com- | | | | | | | | | | |
|---|---|---|---|---|---|---|---|---|---|---|
| pound | X^{1a} | X^{1b} | X^{2a} | X^{2b} | X^{3a} | X^{3b} | X^{4a} | X^{4b} | R¹ | Y |
| 200 | D | D | D | D | D | D | D | D | CD₃ | D |
| 201 | D | D | D | D | D | D | D | D | CD₃ | H |
| 202 | D | D | D | D | D | D | D | D | CH₃ | D |
| 203 | D | D | D | D | D | D | D | D | CH₃ | H |
| 204 | H | H | D | D | D | D | H | H | CD₃ | D |
| 205 | H | H | D | D | D | D | H | H | CD₃ | H |
| 206 | H | H | D | D | D | D | H | H | CH₃ | D |
| 207 | H | H | D | D | D | D | H | H | CH₃ | H |
| 208 | D | D | H | H | H | H | D | D | CD₃ | D |
| 209 | D | D | H | H | H | H | D | D | CD₃ | H |
| 210 | D | D | H | H | H | H | D | D | CH₃ | D |
| 211 | D | D | H | H | H | H | D | D | CH₃ | H |
| 212 | D | D | D | D | H | H | H | H | CD₃ | D |
| 213 | D | D | D | D | H | H | H | H | CD₃ | H |
| 214 | D | D | D | D | H | H | H | H | CH₃ | D |
| 215 | D | D | D | D | H | H | H | H | CH₃ | H |
| 216 | H | H | H | H | D | D | D | D | CD₃ | D |
| 217 | H | H | H | H | D | D | D | D | CD₃ | H |
| 218 | H | H | H | H | D | D | D | D | CH₃ | D |
| 219 | H | H | H | H | D | D | D | D | CH₃ | H |
| 220 | D | D | H | H | D | D | H | H | CD₃ | D |
| 221 | D | D | H | H | D | D | H | H | CD₃ | H |
| 222 | D | D | H | H | D | D | H | H | CH₃ | D |
| 223 | D | D | H | H | D | D | H | H | CH₃ | H |
| 224 | H | H | D | D | H | H | D | D | CD₃ | D |
| 225 | H | H | D | D | H | H | D | D | CD₃ | H |
| 226 | H | H | D | D | H | H | D | D | CH₃ | D |
| 227 | H | H | D | D | H | H | D | D | CH₃ | H |
| 228 | D | D | H | H | H | H | H | H | CD₃ | D |
| 229 | D | D | H | H | H | H | H | H | CD₃ | H |
| 230 | D | D | H | H | H | H | H | H | CH₃ | D |
| 231 | D | D | H | H | H | H | H | H | CH₃ | H |
| 232 | H | H | H | H | H | H | D | D | CD₃ | D |
| 233 | H | H | H | H | H | H | D | D | CD₃ | H |
| 234 | H | H | H | H | H | H | D | D | CH₃ | D |
| 235 | H | H | H | H | H | H | D | D | CH₃ | H |
| 236 | H | H | H | H | H | H | H | H | CD₃ | D |
| 237 | H | H | H | H | H | H | H | H | CD₃ | H |
| 238 | H | H | H | H | H | H | H | H | CH₃ | D |

or a pharmaceutically acceptable salt of any of the foregoing compounds.

[55] In another embodiment, the compound of Formula Ia is a compound wherein Z is —NHSO₂CD₃ and X^{1a,} X^{1b}, X^{2a}, X^{2b}, X^{3a}, X^{3b}, X^{4a}, X^{4b}, R¹ and Y are as shown in Table 3:

TABLE 3
Examples of Compounds of Formula
Ia wherein Z is —NHSO₂CD₃.

| Com- | | | | | | | | | | |
|---|---|---|---|---|---|---|---|---|---|---|
| pound | X^{1a} | X^{1b} | X^{2a} | X^{2b} | X^{3a} | X^{3b} | X^{4a} | X^{4b} | R¹ | Y |
| 300 | D | D | D | D | D | D | D | D | CD₃ | D |
| 301 | D | D | D | D | D | D | D | D | CD₃ | H |
| 302 | D | D | D | D | D | D | D | D | CH₃ | D |
| 303 | D | D | D | D | D | D | D | D | CH₃ | H |
| 304 | H | H | D | D | D | D | H | H | CD₃ | D |
| 305 | H | H | D | D | D | D | H | H | CD₃ | H |
| 306 | H | H | D | D | D | D | H | H | CH₃ | D |
| 307 | H | H | D | D | D | D | H | H | CH₃ | H |
| 308 | D | D | H | H | H | H | D | D | CD₃ | D |
| 309 | D | D | H | H | H | H | D | D | CD₃ | H |
| 310 | D | D | H | H | H | H | D | D | CH₃ | D |
| 311 | D | D | H | H | H | H | D | D | CH₃ | H |
| 312 | D | D | D | D | H | H | H | H | CD₃ | D |
| 313 | D | D | D | D | H | H | H | H | CD₃ | H |
| 314 | D | D | D | D | H | H | H | H | CH₃ | D |
| 315 | D | D | D | D | H | H | H | H | CH₃ | H |
| 316 | H | H | H | H | D | D | D | D | CD₃ | D |
| 317 | H | H | H | H | D | D | D | D | CD₃ | H |
| 318 | H | H | H | H | D | D | D | D | CH₃ | D |
| 319 | H | H | H | H | D | D | D | D | CH₃ | H |
| 320 | D | D | H | H | D | D | H | H | CD₃ | D |
| 321 | D | D | H | H | D | D | H | H | CD₃ | H |
| 322 | D | D | H | H | D | D | H | H | CH₃ | D |
| 323 | D | D | H | H | D | D | H | H | CH₃ | H |
| 324 | H | H | D | D | H | H | D | D | CD₃ | D |
| 325 | H | H | D | D | H | H | D | D | CD₃ | H |
| 326 | H | H | D | D | H | H | D | D | CH₃ | D |
| 327 | H | H | D | D | H | H | D | D | CH₃ | H |
| 328 | D | D | H | H | H | H | H | H | CD₃ | D |
| 329 | D | D | H | H | H | H | H | H | CD₃ | H |
| 330 | D | D | H | H | H | H | H | H | CH₃ | D |
| 331 | D | D | H | H | H | H | H | H | CH₃ | H |
| 332 | H | H | H | H | H | H | D | D | CD₃ | D |
| 333 | H | H | H | H | H | H | D | D | CD₃ | H |
| 334 | H | H | H | H | H | H | D | D | CH₃ | D |
| 335 | H | H | H | H | H | H | D | D | CH₃ | H |
| 336 | H | H | H | H | H | H | H | H | CD₃ | D |
| 337 | H | H | H | H | H | H | H | H | CD₃ | H |
| 338 | H | H | H | H | H | H | H | H | CH₃ | D |

or a pharmaceutically acceptable salt of any of the foregoing compounds.

In one embodiment, the compound of Formula I is a compound of Formula (Ib′):

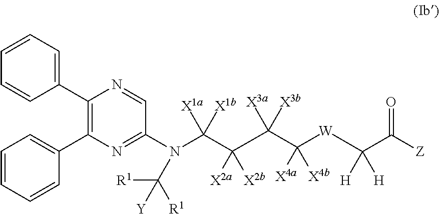

or a pharmaceutically acceptable salt thereof, wherein each X, each R¹, Y and Z are as defined for Formula I.

In one embodiment, the compound of Formula I is a compound of Formula (Ib):

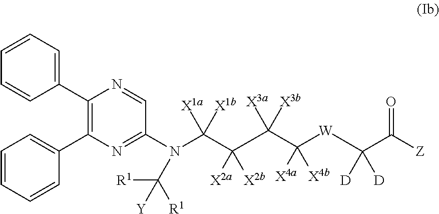

or a pharmaceutically acceptable salt thereof, wherein each X, each R¹, Y and Z are as defined for Formula (I). Exemplary values for the variables in Formula (Ib) and (Ib′) are as provided for Formula (I) and as provided below.

In another set of embodiments, any atom not designated as deuterium in any of the embodiments set forth above is present at its natural isotopic abundance.

In one embodiment, the compound of Formula Ib or Ib′ is a compound wherein Z is —OH and X^{1a,} X^{1b}, X^{2a}, X^{2b}, X^{3a}, X^{3b}, X^{4a}, X^{4b}, R¹ and Y are as shown in Table 4 below.

In one embodiment, the compound of Formula Ib or Ib′ is a compound wherein Z is —NHSO₂CH₃ and X^{1a,} X^{1b}, X^{2a}, X^{2b}, X^{3a}, X^{3b}, X^{4a}, X^{4b}, R¹ and Y are as shown in Table 5 below.

In one embodiment, the compound of Formula Ib or Ib′ is a compound wherein Z is —NHSO₂CD₃ and X^{1a,} X^{1b}, X^{2a}, X^{2b}, X^{3a}, X^{3b}, X^{4a}, X^{4b}, R¹ and Y are as shown in Table 6 below.

In each of tables 4, 5, and 6, the endings “b” and “b′” refer to compounds of Formula Ib and Ib′, respectively.

TABLE 4
Examples of Compounds of Formula Ib or Ib′
wherein Z is —OH, wherein any atom not designated
as deuterium is present at its natural isotopic abundance.

| Com- | | | | | | | | | | |
|---|---|---|---|---|---|---|---|---|---|---|
| pound | X^{1a} | X^{1b} | X^{2a} | X^{2b} | X^{3a} | X^{3b} | X^{4a} | X^{4b} | R¹ | Y |
| 400b | D | D | D | D | D | D | D | D | CD₃ | D |
| 400b′ | | | | | | | | | | |
| 401b | D | D | D | D | D | D | D | D | CD₃ | H |
| 401b′ | | | | | | | | | | |
| 402b | D | D | D | D | D | D | D | D | CH₃ | D |
| 402b′ | | | | | | | | | | |
| 403b | D | D | D | D | D | D | D | D | CH₃ | H |
| 403b′ | | | | | | | | | | |
| 404b | H | H | D | D | D | D | H | H | CD₃ | D |
| 404b′ | | | | | | | | | | |
| 405b | H | H | D | D | D | D | H | H | CD₃ | H |
| 405b′ | | | | | | | | | | |
| 406b | H | H | D | D | D | D | H | H | CH₃ | D |
| 406b′ | | | | | | | | | | |
| 407b | H | H | D | D | D | D | H | H | CH₃ | H |
| 407b′ | | | | | | | | | | |
| 408b | D | D | H | H | H | H | D | D | CD₃ | D |
| 408b′ | | | | | | | | | | |
| 409b | D | D | H | H | H | H | D | D | CD₃ | H |
| 409b′ | | | | | | | | | | |
| 410b | D | D | H | H | H | H | D | D | CH₃ | D |
| 410b′ | | | | | | | | | | |
| 411b | D | D | H | H | H | H | D | D | CH₃ | H |
| 411b′ | | | | | | | | | | |
| 412b | D | D | D | D | H | H | H | H | CD₃ | D |
| 412b′ | | | | | | | | | | |
| 413b | D | D | D | D | H | H | H | H | CD₃ | H |
| 413b′ | | | | | | | | | | |
| 414b | D | D | D | D | H | H | H | H | CH₃ | D |
| 414b′ | | | | | | | | | | |
| 415b | D | D | D | D | H | H | H | H | CH₃ | H |
| 415b′ | | | | | | | | | | |
| 416b | H | H | H | H | D | D | D | D | CD₃ | D |
| 416b′ | | | | | | | | | | |
| 417b | H | H | H | H | D | D | D | D | CD₃ | H |
| 417b′ | | | | | | | | | | |
| 418b | H | H | H | H | D | D | D | D | CH₃ | D |
| 418b′ | | | | | | | | | | |
| 419b | H | H | H | H | D | D | D | D | CH₃ | H |
| 419b′ | | | | | | | | | | |
| 420b | D | D | H | H | D | D | H | H | CD₃ | D |
| 420b′ | | | | | | | | | | |
| 421b | D | D | H | H | D | D | H | H | CD₃ | H |
| 421b′ | | | | | | | | | | |
| 422b | D | D | H | H | D | D | H | H | CH₃ | D |
| 422b′ | | | | | | | | | | |
| 423b | D | D | H | H | D | D | H | H | CH₃ | H |
| 423b′ | | | | | | | | | | |
| 424b | H | H | D | D | H | H | D | D | CD₃ | D |
| 424b′ | | | | | | | | | | |
| 425b | H | H | D | D | H | H | D | D | CD₃ | H |
| 425b′ | | | | | | | | | | |
| 426b | H | H | D | D | H | H | D | D | CH₃ | D |
| 426b′ | | | | | | | | | | |
| 427b | H | H | D | D | H | H | D | D | CH₃ | H |
| 427b′ | | | | | | | | | | |
| 428b | D | D | H | H | H | H | H | H | CD₃ | D |
| 428b′ | | | | | | | | | | |
| 429b | D | D | H | H | H | H | H | H | CD₃ | H |
| 429b′ | | | | | | | | | | |
| 430b | D | D | H | H | H | H | H | H | CH₃ | D |
| 430b′ | | | | | | | | | | |
| 431b | D | D | H | H | H | H | H | H | CH₃ | H |
| 431b′ | | | | | | | | | | |
| 432b | H | H | H | H | H | H | D | D | CD₃ | D |
| 432b′ | | | | | | | | | | |
| 433b | H | H | H | H | H | H | D | D | CD₃ | H |
| 432b′ | | | | | | | | | | |
| 434b | H | H | H | H | H | H | D | D | CH₃ | D |
| 434b′ | | | | | | | | | | |
| 435b | H | H | H | H | H | H | D | D | CH₃ | H |
| 435b′ | | | | | | | | | | |
| 436b | H | H | H | H | H | H | H | H | CD₃ | D |
| 436b′ | | | | | | | | | | |
| 437b | H | H | H | H | H | H | H | H | CD₃ | H |
| 437b′ | | | | | | | | | | |
| 438b | H | H | H | H | H | H | H | H | CH₃ | D |
| 438b′ | | | | | | | | | | |

or a pharmaceutically acceptable salt of any of the foregoing compounds.

In another embodiment, the compound of Formula Ib or Ib′ is a compound wherein Z is —NHSO₂CH₃ and X^{1a,} X^{1b}, X^{2a}, X^{2b}, X^{3a}, X^{3b}, X^{4a}, X^{4b}, R¹ and Y are as shown in Table 5, wherein any atom not designated as deuterium is present at its natural isotopic abundance:

TABLE 5
Examples of Compounds of Formula
Ib or Ib′ wherein Z is —NHSO₂CH₃.

| Com- | | | | | | | | | | |
|---|---|---|---|---|---|---|---|---|---|---|
| pound | X^{1a} | X^{1b} | X^{2a} | X^{2b} | X^{3a} | X^{3b} | X^{4a} | X^{4b} | R¹ | Y |
| 500b | D | D | D | D | D | D | D | D | CD₃ | D |
| 500b′ | | | | | | | | | | |
| 501b | D | D | D | D | D | D | D | D | CD₃ | H |
| 501b′ | | | | | | | | | | |
| 502b | D | D | D | D | D | D | D | D | CH₃ | D |
| 502b′ | | | | | | | | | | |
| 503b | D | D | D | D | D | D | D | D | CH₃ | H |
| 503b′ | | | | | | | | | | |
| 504b | H | H | D | D | D | D | H | H | CD₃ | D |
| 504b′ | | | | | | | | | | |
| 505b | H | H | D | D | D | D | H | H | CD₃ | H |
| 505b′ | | | | | | | | | | |
| 506b | H | H | D | D | D | D | H | H | CH₃ | D |
| 506b′ | | | | | | | | | | |
| 507b | H | H | D | D | D | D | H | H | CH₃ | H |
| 507b′ | | | | | | | | | | |
| 508b | D | D | H | H | H | H | D | D | CD₃ | D |
| 508b′ | | | | | | | | | | |
| 509b | D | D | H | H | H | H | D | D | CD₃ | H |
| 509b′ | | | | | | | | | | |
| 510b | D | D | H | H | H | H | D | D | CH₃ | D |
| 510b′ | | | | | | | | | | |
| 511b | D | D | H | H | H | H | D | D | CH₃ | H |
| 511b′ | | | | | | | | | | |
| 512b | D | D | D | D | H | H | H | H | CD₃ | D |
| 512b′ | | | | | | | | | | |
| 513b | D | D | D | D | H | H | H | H | CD₃ | H |
| 513b′ | | | | | | | | | | |
| 514b | D | D | D | D | H | H | H | H | CH₃ | D |
| 514b′ | | | | | | | | | | |
| 515b | D | D | D | D | H | H | H | H | CH₃ | H |
| 515b′ | | | | | | | | | | |
| 516b | H | H | H | H | D | D | D | D | CD₃ | D |
| 516b′ | | | | | | | | | | |
| 517b | H | H | H | H | D | D | D | D | CD₃ | H |
| 517b′ | | | | | | | | | | |
| 518b | H | H | H | H | D | D | D | D | CH₃ | D |
| 518b′ | | | | | | | | | | |
| 519b | H | H | H | H | D | D | D | D | CH₃ | H |
| 519b′ | | | | | | | | | | |
| 520b | D | D | H | H | D | D | H | H | CD₃ | D |
| 520b′ | | | | | | | | | | |
| 521b | D | D | H | H | D | D | H | H | CD₃ | H |
| 521b′ | | | | | | | | | | |
| 522b | D | D | H | H | D | D | H | H | CH₃ | D |
| 522b′ | | | | | | | | | | |
| 523b | D | D | H | H | D | D | H | H | CH₃ | H |
| 523b′ | | | | | | | | | | |
| 524b | H | H | D | D | H | H | D | D | CD₃ | D |
| 524b′ | | | | | | | | | | |
| 525b | H | H | D | D | H | H | D | D | CD₃ | H |
| 525b′ | | | | | | | | | | |
| 526b | H | H | D | D | H | H | D | D | CH₃ | D |
| 526b′ | | | | | | | | | | |
| 527b | H | H | D | D | H | H | D | D | CH₃ | H |
| 527b′ | | | | | | | | | | |
| 528b | D | D | H | H | H | H | H | H | CD₃ | D |
| 528b′ | | | | | | | | | | |
| 529b | D | D | H | H | H | H | H | H | CD₃ | H |
| 529b′ | | | | | | | | | | |
| 530b | D | D | H | H | H | H | H | H | CH₃ | D |
| 530b′ | | | | | | | | | | |
| 531b | D | D | H | H | H | H | H | H | CH₃ | H |
| 531b′ | | | | | | | | | | |
| 532b | H | H | H | H | H | H | D | D | CD₃ | D |
| 532b′ | | | | | | | | | | |
| 533b | H | H | H | H | H | H | D | D | CD₃ | H |
| 533b′ | | | | | | | | | | |
| 534b | H | H | H | H | H | H | D | D | CH₃ | D |
| 534b′ | | | | | | | | | | |
| 535b | H | H | H | H | H | H | D | D | CH₃ | H |
| 535b′ | | | | | | | | | | |
| 536b | H | H | H | H | H | H | H | H | CD₃ | D |
| 536b′ | | | | | | | | | | |
| 537b | H | H | H | H | H | H | H | H | CD₃ | H |
| 537b′ | | | | | | | | | | |
| 538b | H | H | H | H | H | H | H | H | CH₃ | D |
| 538b′ | | | | | | | | | | |

or a pharmaceutically acceptable salt of any of the foregoing compounds.

In another embodiment, the compound of Formula Ib or Ib′ is a compound wherein Z is —NHSO₂CD₃ and X^{1a,} X^{1b}, X^{2a}, X^{2b}, X^{3a}, X^{3b}, X^{4a}, X^{4b}, R¹ and Y are as shown in Table 6, wherein any atom not designated as deuterium is present at its natural isotopic abundance:

TABLE 6
Examples of Compounds of Formula Ib
or Ib′ wherein Z is —NHSO₂CD₃.

| Compound | X^{1a} | X^{1b} | X^{2a} | X^{2b} | X^{3a} | X^{3b} | X^{4a} | X^{4b} | R¹ | Y |
|---|---|---|---|---|---|---|---|---|---|---|
| 600b | D | D | D | D | D | D | D | D | CD₃ | D |
| 600b′ | | | | | | | | | | |
| 601b | D | D | D | D | D | D | D | D | CD₃ | H |
| 601b′ | | | | | | | | | | |
| 602b | D | D | D | D | D | D | D | D | CH₃ | D |
| 602b′ | | | | | | | | | | |
| 603b | D | D | D | D | D | D | D | D | CH₃ | H |
| 603b′ | | | | | | | | | | |
| 604b | H | H | D | D | D | D | H | H | CD₃ | D |
| 604b′ | | | | | | | | | | |
| 605b | H | H | D | D | D | D | H | H | CD₃ | H |
| 605b′ | | | | | | | | | | |
| 606b | H | H | D | D | D | D | H | H | CH₃ | D |
| 606b′ | | | | | | | | | | |
| 607b | H | H | D | D | D | D | H | H | CH₃ | H |
| 607b′ | | | | | | | | | | |
| 608b | D | D | H | H | H | H | D | D | CD₃ | D |
| 608b′ | | | | | | | | | | |
| 609b | D | D | H | H | H | H | D | D | CD₃ | H |
| 609b′ | | | | | | | | | | |
| 610b | D | D | H | H | H | H | D | D | CH₃ | D |
| 610b′ | | | | | | | | | | |
| 611b | D | D | H | H | H | H | D | D | CH₃ | H |
| 611b′ | | | | | | | | | | |
| 612b | D | D | D | D | H | H | H | H | CD₃ | D |
| 612b′ | | | | | | | | | | |
| 613b | D | D | D | D | H | H | H | H | CD₃ | H |
| 613b′ | | | | | | | | | | |
| 614b | D | D | D | D | H | H | H | H | CH₃ | D |
| 614b′ | | | | | | | | | | |
| 615b | D | D | D | D | H | H | H | H | CH₃ | H |
| 615b′ | | | | | | | | | | |
| 616b | H | H | H | H | D | D | D | D | CD₃ | D |
| 616b′ | | | | | | | | | | |
| 617b | H | H | H | H | D | D | D | D | CD₃ | H |
| 617b′ | | | | | | | | | | |
| 618b | H | H | H | H | D | D | D | D | CH₃ | D |
| 618b′ | | | | | | | | | | |
| 619b | H | H | H | H | D | D | D | D | CH₃ | H |
| 619b′ | | | | | | | | | | |
| 620b | D | D | H | H | D | D | H | H | CD₃ | D |
| 620b′ | | | | | | | | | | |
| 621b | D | D | H | H | D | D | H | H | CD₃ | H |
| 621b′ | | | | | | | | | | |
| 622b | D | D | H | H | D | D | H | H | CH₃ | D |
| 622b′ | | | | | | | | | | |
| 623b | D | D | H | H | D | D | H | H | CH₃ | H |
| 623b′ | | | | | | | | | | |
| 624b | H | H | D | D | H | H | D | D | CD₃ | D |
| 624b′ | | | | | | | | | | |
| 625b | H | H | D | D | H | H | D | D | CD₃ | H |
| 625b′ | | | | | | | | | | |
| 626b | H | H | D | D | H | H | D | D | CH₃ | D |
| 626b′ | | | | | | | | | | |
| 627b | H | H | D | D | H | H | D | D | CH₃ | H |
| 627b′ | | | | | | | | | | |
| 628b | D | D | H | H | H | H | H | H | CD₃ | D |
| 628b′ | | | | | | | | | | |
| 629b | D | D | H | H | H | H | H | H | CD₃ | H |
| 629b′ | | | | | | | | | | |
| 630b | D | D | H | H | H | H | H | H | CH₃ | D |
| 630b′ | | | | | | | | | | |
| 631b | D | D | H | H | H | H | H | H | CH₃ | H |
| 631b′ | | | | | | | | | | |
| 632b | H | H | H | H | H | H | D | D | CD₃ | D |
| 623b′ | | | | | | | | | | |
| 633b | H | H | H | H | H | H | D | D | CD₃ | H |
| 633b′ | | | | | | | | | | |
| 634b | H | H | H | H | H | H | D | D | CH₃ | D |
| 634b′ | | | | | | | | | | |
| 635b | H | H | H | H | H | H | D | D | CH₃ | H |
| 635b′ | | | | | | | | | | |
| 636b | H | H | H | H | H | H | H | H | CD₃ | D |
| 636b′ | | | | | | | | | | |
| 637b | H | H | H | H | H | H | H | H | CD₃ | H |
| 637b′ | | | | | | | | | | |
| 638b | H | H | H | H | H | H | H | H | CH₃ | D |
| 638b′ | | | | | | | | | | |

or a pharmaceutically acceptable salt of any of the foregoing compounds.

In another embodiment, the compound of the invention is compound 400b′, 500b′, 403b′, 503b′, 436b′, or 536b′ herein, or a pharmaceutically acceptable salt of any of the foregoing compounds.

The synthesis of compounds of Formula I, including compounds of Formula Ia, Formula Ib′ and Formula Ib, can be readily achieved by synthetic chemists of ordinary skill by reference to the Exemplary Synthesis disclosed herein.

### EXEMPLARY SYNTHESIS

The syntheses of MRE-269 and NS-304 are described in the following publications and patent: Asaki, T., et al., Bioorganic and Medicinal Chemistry, 2007, 15: 6692; Asaki, T., et al., Bioorganic and Medicinal Chemistry Letters, 2007, 17: 6588; Asaki, T., et al., Bioorganic and Medicinal Chemistry, 2007, 15: 7720; U.S. Pat. No. 7,205,302 B2. Compounds of Formula I may be prepared in an analogous manner by using appropriately deuterated reagents and/or intermediates according to the general procedures shown in Schemes 1-4. In the schemes, “Ph” is used to denote phenyl

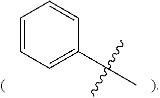 [SMILES: *c1ccccc1]

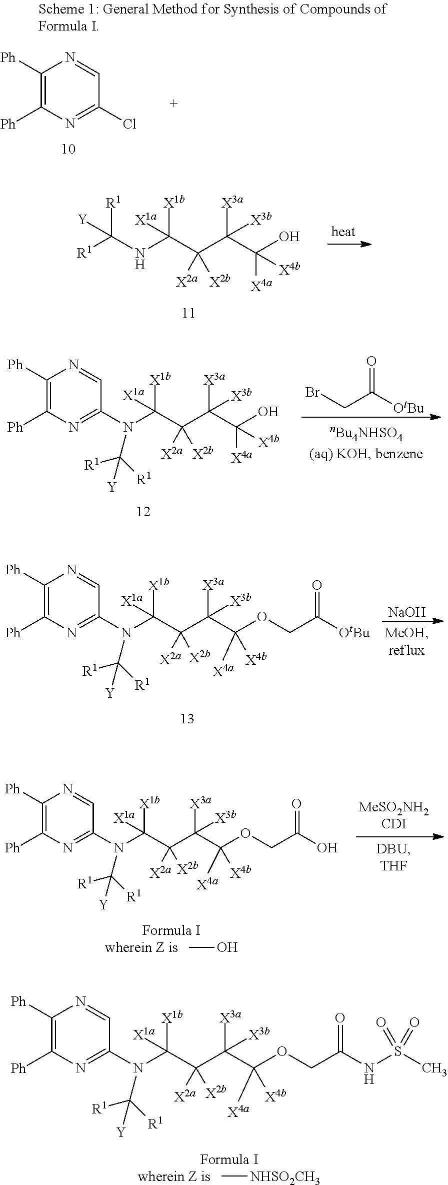

Scheme 1 shows a convenient method for synthesizing compounds of Formula I. As depicted in Scheme 1 above, treatment of commercially available 5-chloro-2,3-diphenylpyrazine 10 with appropriately deuterated 4-amino-butan-1-ol derivative 11 under thermal conditions according to the procedure described by Asaki, T., et al., Bioorganic and Medicinal Chemistry, 2007, 15: 6692-6704 affords appropriately deuterated aminopyrazine 12. Following the general protocols of Asaki, subsequent O-alkylation of the primary alcohol with commercially available tert-butyl bromoacetate in the presence of aqueous potassium hydroxide and tetrabutylammonium bisulfate gives pyrazinyl tert-butyl ester 13. Saponification of the tert-butyl ester 13 with methanolic sodium hydroxide provides compounds of Formula I wherein Z is —OH. Treatment of the carboxylic acid with commercially available methanesulfonamide in the presence of 1,1′-carbonyldiimidazole (CDI) and 1,8-diazabicyclo[5.4.0]undec-7-ene (DBU) according to the general procedure described by Asaki, T., et al., Bioorganic and Medicinal Chemistry, 2007, 15: 7720-7725 affords compounds of Formula I wherein Z is —NHSO₂CH₃.

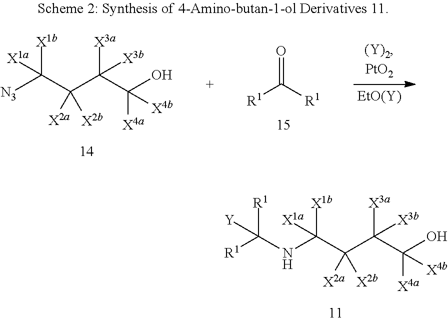

The synthesis of appropriately deuterated 4-amino-butan-1-ol derivatives 11 is shown in Scheme 2. Treatment of deuterated 4-azido-butan-1-ol 14 with deuterated acetone 15 in the presence of either deuterium or hydrogen gas and platinum (IV) oxide according to the general procedure described by Reinfried, R. et al., Canadian Journal of Chemistry, 1974, 52: 4083-4089 gives appropriately deuterated 4-amino-butan-1-ol derivative 11. As an example of deuterated acetone 15, deuterated reagent 15a is commercially available from CDN Isotopes Inc.:

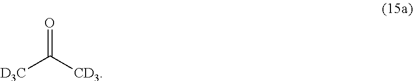

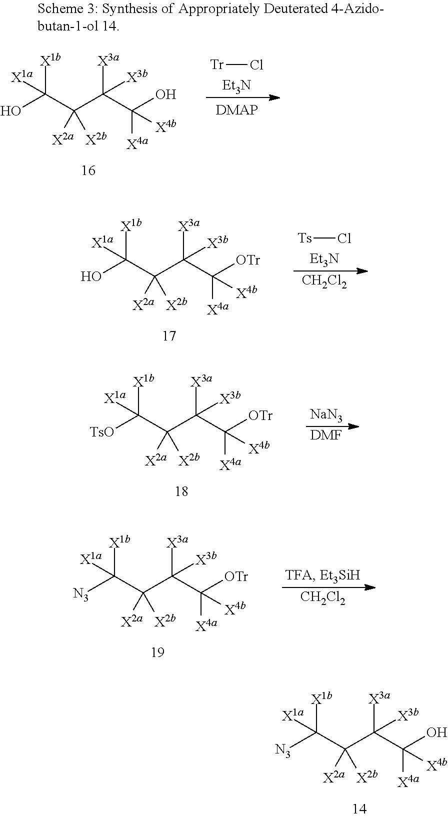

The synthesis of appropriately deuterated 4-azido-butan-1-ol 14 is shown in Scheme 3. Treatment of appropriately deuterated 1,4-butanediol 16 with trityl chloride (Tr-Cl) in the presence of triethylamine in a manner analogous to the procedure described for d8-1,4-butanediol by Overkleeft, H. S. et al., Bioorganic and Medicinal Chemistry Letters, 2004, 14: 3131-3134 gives monotrityl ether 17. The following examples of deuterated 1,4-butanediol 16 are commercially available from CDN Isotopes Inc.:

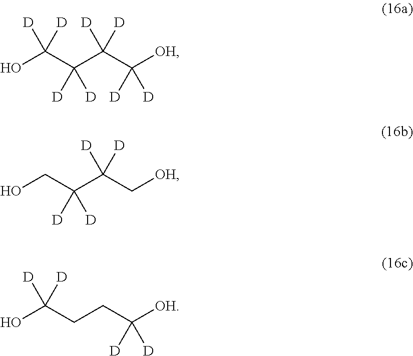 [SMILES: [2H]C([2H])(O)C([2H])([2H])C([2H])([2H])C([2H])([2H])O ; [2H]C([2H])(CO)C([2H])([2H])CO ; [2H]C([2H])(O)CCC([2H])([2H])O]

Following the general protocols from Overkleeft, tosylation of the remaining primary alcohol in the presence of triethylamine followed by displacement of the derived tosylate 18 with sodium azide gives the appropriately deuterated azido-trityl ether 19. Finally, deprotection of the trityl ether via the Overkleeft procedure (trifluoroacetic acid and triethylsilane) affords appropriately-deuterated 4-azido-butan-1-ol 14.

The synthetic routes to several partially-deuterated 1,4-butanediol intermediates 16 are shown in Schemes 4a, 4b, and 4c, below.

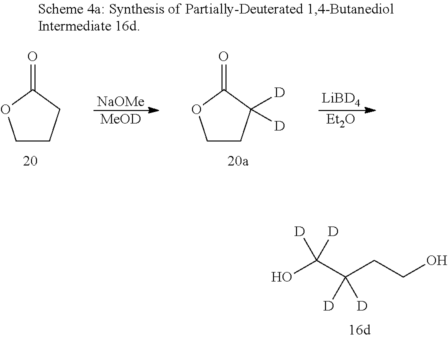

As depicted in Scheme 4a, the preparation of 1,1,2,2-tetradeutero-1,4-butanediol 16d involves treatment of commercially available gamma-butyrolactone 20 with sodium methoxide in d1-methanol according to the procedure described by Keay, B. A. et al., Journal of Organic Chemistry, 2007, 72: 7253-7259 to give d2-lactone 20a. Reduction of the lactone with commercially available lithium borodeuteride according to the procedure described by Brown, H. C. et al., Journal of Organic Chemistry, 1982, 47, pp 4702-4708 affords desired 1,1,2,2-tetradeutero-1,4-butanediol 16d.

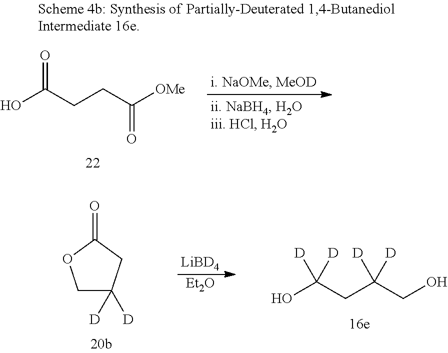

As depicted in Scheme 4b, the preparation of 1,1,3,3-tetradeutero-1,4-butanediol 16e involves base-catalyzed H/D exchange of mono-methyl succinate 22 (prepared according to the procedure described by Keay, B. A. et al., Journal of Organic Chemistry, 2007, 72, pp 7253-7259) followed by selective reduction with sodium borohydride and cyclization under acidic conditions affords the 4,4-dideuterodihydrofuran-2(3H)-one 20b. Reduction of lactone 20b with commercially available lithium borodeuteride according to the procedure described by Brown, H. C. et al., Journal of Organic Chemistry, 1982, 47, pp 4702-4708 affords the desired 1,1,3,3-tetradeutero-1,4-butanediol16e.

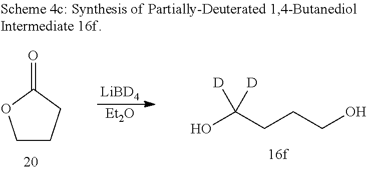

As depicted in Scheme 4c, the preparation of 1,1-dideutero-1,4-butanediol 16f involves reduction of commercially available gamma-butyrolactone 20 with commercially available lithium borodeuteride according to the procedure described by Brown, H. C. et al., Journal of Organic Chemistry, 1982, 47, pp 4702-4708 to afford 1,1-dideutero-1,4-butanediol 16f.

Combinations of substituents and variables envisioned by this invention are only those that result in the formation of stable compounds.

### COMPOSITIONS

The invention also provides pyrogen-free compositions comprising a compound of Formula I (e.g., including any of the formulae herein), or a pharmaceutically acceptable salt of said compound; and an acceptable carrier. In one aspect, the pyrogen-free compositions comprise an effective amount of a compound of Formula I (e.g., including any of the formulae herein), or a pharmaceutically acceptable salt of said compound; and an acceptable carrier. Preferably, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. In one aspect, the composition further comprises an effective amount of a second therapeutic agent. The second therapeutic agent may be selected from any compound or therapeutic agent known to have or that demonstrates advantageous properties when administered with a compound that acts as a PGI₂ receptor agonist. The second agent may be, for example, an agent useful in the inhibition of platelet aggregation, vasodilation, inhibition of lipid deposition, and/or inhibition of leukocyte activation.

Preferably, the second therapeutic agent is an agent useful in the treatment of a disease or condition selected from pulmonary arterial hypertension, peripheral vascular diseases (for example, arteriosclerosis obliterans, intermittent claudication, peripheral arterial embolism, vibration disease, and Raynaud's disease), systemic lupus erythematosus, reocclusion or restenosis after percutaneous transluminal coronary angioplasty (PTCA), arteriosclerosis, thrombosis, diabetic neuropathy, diabetic nephropathy, hypertension, ischemic diseases (for example, cerebral infarction and myocardial infarction), transient ischemic attack and glomerulonephritis, or acceleration of angiogenesis in peripheral blood vessel reconstruction technique or angiogenesis therapy.

In one embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to reduce the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother. Rep 50: 219. Body surface area may be approximately determined from height and weight of the subject, which can be a patient. See, e.g., Scientific Tables, Geigy Pharmaceuticals, Ardsley, N.Y., 1970, 537.

In one embodiment, an effective amount of a compound of this invention can range from about 0.01 to about 5000 mg per treatment. In more specific embodiments the range is from about 0.1 to 2500 mg, or from 0.2 to 1000 mg, or most specifically from about 1 to 500 mg. Treatment typically is administered one to three times daily.

Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### METHODS OF TREATMENT

According to another embodiment, the invention provides a method of treating a subject suffering from, or susceptible to, a disease that is beneficially treated by NS-304, comprising the step of administering to the subject an effective amount of a compound of this invention or a pharmaceutically acceptable salt of said compound or a composition of this invention. In one embodiment the subject is a patient. Such diseases are well known in the art and examples thereof are described in U.S. Pat. No. 7,205,302. Such diseases include diseases that may be treated by inhibition of platelet aggregation, vasodilation, inhibition of lipid deposition, and/or inhibition of leukocyte activation. Such diseases include pulmonary arterial hypertension, peripheral vascular diseases (for example, arteriosclerosis obliterans, intermittent claudication, peripheral arterial embolism, vibration disease, and Raynaud's disease), systemic lupus erythematosus, reocclusion or restenosis after percutaneous transluminal coronary angioplasty (PTCA), arteriosclerosis, thrombosis, diabetic neuropathy, diabetic nephropathy, hypertension, ischemic diseases (for example, cerebral infarction and myocardial infarction), transient ischemic attack and glomerulonephritis, or acceleration of angiogenesis in peripheral blood vessel reconstruction technique or angiogenesis therapy. In a particular embodiment, the disease is selected from pulmonary arterial hypertension, peripheral vascular diseases arteriosclerosis obliterans, intermittent claudication, and peripheral arterial embolism.

Methods delineated herein also include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In another embodiment, any of the above methods of treatment comprises the further step of co-administering to the subject one or more second therapeutic agents. The choice of second therapeutic agent may be made from any second therapeutic agent known to be useful for co-administration with a compound that acts as a PGI₂ receptor agonist The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2^{nd} Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I, or a pharmaceutically acceptable salt of said compound, alone or together with one or more of the above-described second therapeutic agents in the manufacture of a medicament, either as a single composition or as separate dosage forms, for treatment or prevention in a subject of a disease, disorder or symptom set forth above. Another aspect of the invention is a compound of Formula I for use in the treatment or prevention in a subject of a disease, disorder or symptom thereof delineated herein.

### EXAMPLES

### Example 1

### Synthesis of 2-(1,1,2,2,3,3,4,4-d₈-4((5,6-Diphenylpyrazin-2-yl)(perdeutero-propan-2-yl)amino)butoxy)acetic acid (Compound 400b′)

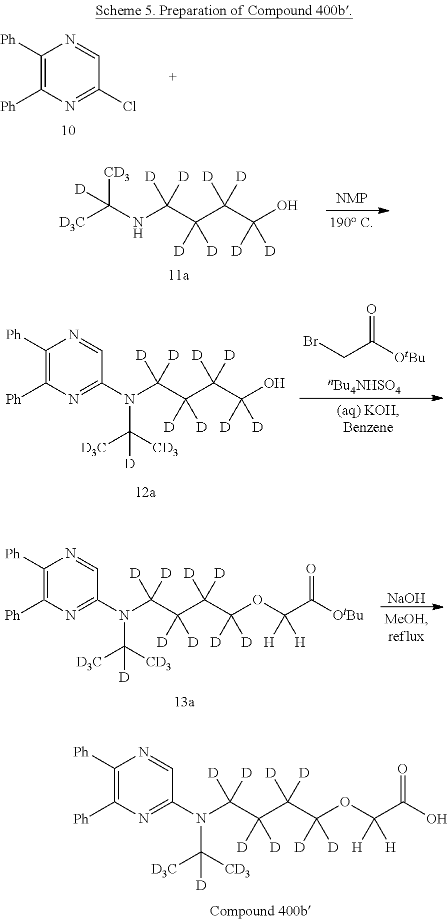

Step 1. 2-(1,1,2,2,3,3,4,4-d₈-4((5,6-Diphenylpyrazin-2-yl)(perdeutero-propan-2-yl)amino)butan-1-ol (12a). To a solution of commercially available 5-chloro-2,3-diphenylpyrazine (10) (0.56 g, 2.10 mmol) in NMP (2.1 mL) was added d₁₅-aminoalcohol 11a (0.46 g, 3.15 mmol, 1.5 equiv, prepared as described in Example 8). The reaction vessel was sealed and heated to 190° C. for 15 hours, then cooled to ambient temperature. The mixture was diluted with ice water and extracted with Et₂O (3×20 mL). The combined organic layers were washed successively with water and brine. The resulting organic layer was dried (MgSO₄), filtered and concentrated in vacuo. The resulting residue was purified by column chromatography (SiO₂, 30-50% EtOAc/heptane) to afford 12a (0.13 g, 16%). MS (M+H): 377.0.

Step 2. tert-Butyl 2-(1,1,2,2,3,3,4,4-d₈-4-((5,6-diphenylpyrazin-2-yl)(perdeutero-propan-2-yl)amino)butoxy)acetate (13a). To a solution of alcohol 12a (64 mg, 0.17 mmol) and tetrabutylammonium bisulfate (30 mg, 0.085 mmol, 0.5 equiv) in benzene (1.7 mL) and aqueous KOH (40%, 1.7 mL) at 5° C. , was added dropwise tert-butylbromoacetate (75 μL, 0.51 mmol, 3.0 equiv). The mixture was stirred vigorously at 5° C. for 45 minutes and then warmed to ambient temperature. The mixture was stirred vigorously at ambient temperature for 1 hour, then diluted with ice water and extracted with Et₂O (3×20 mL). The combined organic layers were washed successively with water and brine. The resulting organic layer was dried (MgSO₄), filtered and concentrated in vacuo. The resulting residue was purified by column chromatography (SiO₂, 30% EtOAc/heptane) to afford ester 13a (80 mg, 96%).

Step 3. 2-(1,1,2,2,3,3,4,4-d₈-4-((5,6-Diphenylpyrazin-2-yl)(perdeutero-propan-2-yl)amino)butoxy)acetic acid (Compound 400b′). To a solution of 13a (80 mg, 0.163 mmol) in MeOH (2 mL), was added 1N sodium hydroxide (1.0 mL). The reaction mixture was heated to reflux under stirring for a period of 1 hour and then cooled to ambient temperature. The resulting solution was concentrated in vacuo and the resulting residue dissolved in water (10 mL). The aqueous solution was then washed with Et₂O (2×10 mL). The aqueous phase was separated and acidified to pH˜2 with 1M HCl. The acidified aqueous layer was extracted with EtOAc (3×15 mL) and the combined organic extracts were dried (MgSO₄), filtered and concentrated in vacuo to afford Compound 400b′ (52 mg, 74%). MS (M+H): 435.0. The crude acid was used directly in the subsequent step.

### Example 2

### Synthesis of N-(Methylsulfonyl)-2-(1,1,2,2,3,3,4,4-d₈-4-((5,6-diphenylpyrazin-2-yl)(perdeutero-propan-2-yl)amino)butoxy)acetamide (Compound 500b′)

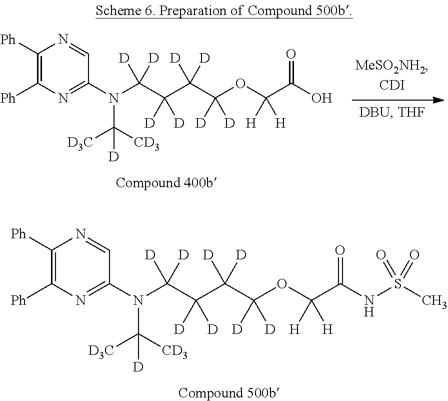 [SMILES: [2H]C([2H])([2H])C([2H])(N(c1cnc(-c2ccccc2)c(-c2ccccc2)n1)C([2H])([2H])C([2H])([2H])C([2H])([2H])C([2H])([2H])OCC(=O)O)C([2H])([2H])[2H] ; [2H]C([2H])([2H])C([2H])(N(c1cnc(-c2ccccc2)c(-c2ccccc2)n1)C([2H])([2H])C([2H])([2H])C([2H])([2H])C([2H])([2H])OCC(=O)NS(C)(=O)=O)C([2H])([2H])[2H]]

N-(Methylsulfonyl)-2-(1,1,2,2,3,3,4,4-d₈-4-((5,6-diphenylpyrazin-2-yl)(perdeutero-propan-2-yl)amino)butoxy)acetamide (Compound 500b′). To a solution of acid 400b′ (52 mg, 0.12 mmol) in THF (1 mL), was added 1,1′-carbonyldiimidazole (CDI, 22 mg, 0.13 mmol, 1.1 equiv) and the solution was stirred at ambient temperature for 30 minutes then heated to reflux for an additional 30 minutes. After cooling the solution to ambient temperature, methanesulfonamide (13 mg, 0.12 mmol, 1.02 equiv) was added and the mixture stirred for 10 minutes at ambient temperature. To the stirred solution, was then added 1,8-diazabicyclo[5.4.0]undec-7-ene (DBU, 20 μL, 0.123 mmol, 1.03 equiv) and the mixture was stirred at ambient temperature for 12 hours then diluted with 1N HCl and extracted with Et₂O (3×20 mL). The combined organic layers were washed successively with water and brine. The resulting organic layer was dried (MgSO₄), filtered and concentrated in vacuo. The resulting residue was purified by column chromatography (SiO₂, 5-10% MeOH/CHCl₃) to afford Compound 500b′ as a pale yellow solid (32 mg, 52%). ¹H NMR (CDCl₃, 400 MHz) δ 8.00 (s, 1H), 7.44 (m, 2H), 7.33 (m, 2H), 7.28-7.21 (m, 6H), 3.97 (s, 2H), 3.29 (s, 3H); MS (M+H) 512.0.

### Example 3

### Synthesis of 2-(1,1,2,2,3,3,4,4-d₈-4-((5,6-Diphenylpyrazin-2-yl)(isopropyl)amino)butoxy)acetic acid (Compound 403b′)

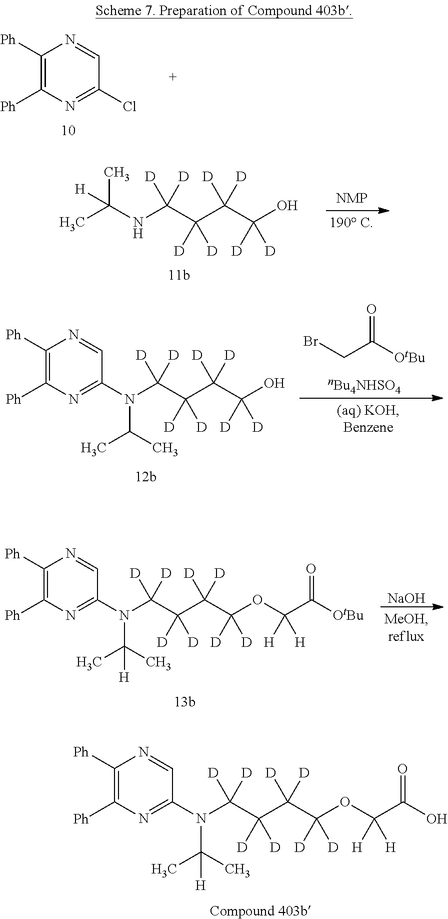

Step 1. 2-(1,1,2,2,3,3,4,4-d₈-4-((5,6-Diphenylpyrazin-2-yl)(propan-2-yl)amino)butan-1-ol (12b). To a solution of commercially available 5-chloro-2,3-diphenylpyrazine (10) (0.57 g, 2.14 mmol) in NMP (2.0 mL), was added d₇-aminoalcohol 11b (0.75 g, 5.35 mmol, 2.5 equiv, prepared as described in Example 10). The reaction vessel was sealed and heated to 190° C. for 15 hours, then cooled to ambient temperature. The mixture was diluted with ice water and extracted with Et₂O (3×20 mL). The combined organic layers were washed successively with water and brine. The resulting organic layer was dried (MgSO₄), filtered and concentrated in vacuo. The resulting residue was purified by column chromatography (SiO₂, 30-50% EtOAc/heptane) to afford 12b (0.20 g, 25%). MS (M+H): 370.0.

Step 2. tert-Butyl 2-(1,1,2,2,3,3,4,4-d₈-4-((5,6-diphenylpyrazin-2-yl)(propan-2-yl)amino)butoxy)acetate (13b). To a solution of alcohol 12b (0.144 g, 0.39 mmol) and tetrabutylammonium bisulfate (66 mg, 0.195 mmol, 0.5 equiv) in benzene (2.0 mL) and aqueous KOH (40%, 2.0 mL) at 5° C. , was added dropwise tert-butylbromoacetate (0.17 mL, 1.17 mmol, 3.0 equiv). The mixture was stirred vigorously at 5° C. for 45 minutes and then warmed to ambient temperature. The mixture was stirred vigorously at ambient temperature for 1 hour, then diluted with ice water and extracted with Et₂O (3×20 mL). The combined organic layers were washed successively with water and brine. The resulting organic layer was dried (MgSO₄), filtered and concentrated in vacuo. The resulting residue was purified by column chromatography (SiO₂, 30% EtOAc/heptane) to afford ester 13b (0.15 g, 79%).

Step 3. 2-(1,1,2,2,3,3,4,4-d₈-4-((5,6-Diphenylpyrazin-2-yl)(propan-2-yl)amino)butoxy)acetic acid (Compound 403b′). To a solution of 13b (0.15 g, 0.31 mmol) in MeOH (3.1 mL), was added 1N sodium hydroxide (1.5 mL). The reaction mixture was heated to reflux under stirring for a period of 1 hour and then cooled to ambient temperature. The resulting solution was concentrated in vacuo and the resulting residue dissolved in water (10 mL). The aqueous solution was then washed with Et₂O (2×10 mL). The aqueous phase was separated and acidified to pH˜2 with 1M HCl. The acidified aqueous layer was extracted with EtOAc (3×15 mL) and the combined organic extracts were dried (MgSO₄), filtered, and concentrated in vacuo to afford Compound 403b′ (0.11 g, 84%). MS (M+H) 428.0. The crude acid was used directly in the subsequent step.

### Example 4

### Synthesis of N-(Methylsulfonyl)-2-(1,1,2,2,3,3,4,4-d₈-4-((5,6-diphenylpyrazin-2-yl)(isopropyl)amino)butoxy)acetamide (Compound 503b′)

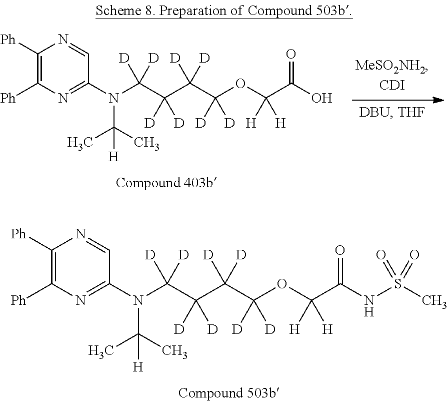 [SMILES: [2H]C([2H])(OCC(=O)O)C([2H])([2H])C([2H])([2H])C([2H])([2H])N(c1cnc(-c2ccccc2)c(-c2ccccc2)n1)C(C)C ; [2H]C([2H])(OCC(=O)NS(C)(=O)=O)C([2H])([2H])C([2H])([2H])C([2H])([2H])N(c1cnc(-c2ccccc2)c(-c2ccccc2)n1)C(C)C]

N-(Methylsulfonyl)-2-(1,1,2,2,3,3,4,4-d₈-4-((5,6-diphenylpyrazin-2-yl)(propan-2-yl)amino)butoxy)acetamide (Compound 503b′). To a solution of acid 403b′ (0.11 g, 0.26 mmol) in THF (3 mL), was added 1,1′-carbonyldiimidazole (CDI, 46 mg, 0.28 mmol, 1.1 equiv) and the solution was stirred at ambient temperature for 30 minutes then heated to reflux for an additional 30 minutes. After cooling the solution to ambient temperature, methanesulfonamide (25 mg, 0.26 mmol, 1.02 equiv) was added and the mixture stirred for 10 minutes at ambient temperature. To the stirred solution, was then added 1,8-diazabicyclo[5.4.0]undec-7-ene (DBU, 40 μL, 0.27 mmol, 1.03 equiv) and the mixture was stirred at ambient temperature for 12 hours then diluted with 1N HCl and extracted with Et₂O (3×20 mL). The combined organic layers were washed successively with water and brine. The resulting organic layer was dried (MgSO₄), filtered, and concentrated in vacuo. The resulting residue was purified by column chromatography (SiO₂, 5-10% MeOH/CHCl₃) to afford Compound 503b′ as a pale yellow solid (73 mg, 56%). ¹H NMR (CDCl₃, 400 MHz) δ 8.00 (s, 1H), 7.44 (m, 2H), 7.35 (m, 2H), 7.30-7.21 (m, 6H), 4.72 (m, 1H), 3.97 (s, 2H), 3.29 (s, 3H), 1.28 (d, J=6.8 Hz, 6H); MS (M+H): 505.3.

### Example 5

### Synthesis of 2-(4-((5,6-Diphenylpyrazin-2-yl)(perdeutero-propan-2-yl)amino)butoxy)acetic acid (Compound 436b′)

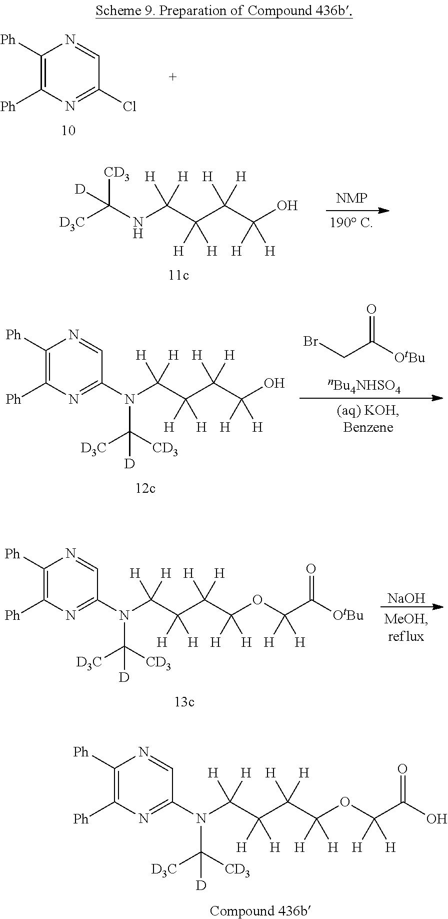

Step 1. 2-(4-((5,6-Diphenylpyrazin-2-yl)(perdeutero-propan-2-yl)amino)butan-1-ol (12c). To a solution of commercially available 5-chloro-2,3-diphenylpyrazine (10) (0.70 g, 2.62 mmol) in NMP (2 mL), was added d₇-aminoalcohol 11c (0.54 g, 3.93 mmol, 1.5 equiv, prepared as described in Example 7). The reaction vessel was sealed and heated to 190° C. for 15 hours, then cooled to ambient temperature. The mixture was diluted with ice water and extracted with Et₂O (3×20 mL). The combined organic layers were washed successively with water and brine. The resulting organic layer was dried (MgSO₄), filtered and concentrated in vacuo. The resulting residue was purified by column chromatography (SiO₂, 30-50% EtOAc/heptane) to afford 12c (0.20 g, 21%). MS (M+H): 369.2.

Step 2. tert-Butyl 2-(4-((5,6-diphenylpyrazin-2-yl)(perdeutero-propan-2-yl)amino)butoxy)acetate (13c). To a solution of alcohol 12c (0.18 g, 0.49 mmol) and tetrabutylammonium bisulfate (42 mg, 0.125 mmol, 0.5 equiv) in benzene (2 mL) and aqueous KOH (40%, 2 mL) at 5° C. , was added dropwise tert-butylbromoacetate (0.05 mL, 0.30 mmol, 1.20 equiv). The mixture was stirred vigorously at 5° C. for 45 minutes and then warmed to ambient temperature. The mixture was stirred vigorously at ambient temperature for 1 hour, then diluted with ice water and extracted with Et₂O (3×20 mL). The combined organic layers were washed successively with water and brine. The resulting organic layer was dried (MgSO₄), filtered and concentrated in vacuo. The resulting residue was purified by column chromatography (SiO₂, 30% EtOAc/heptane) to afford ester 13c (0.21 g, 90%).

Step 3. 2-(4-((5,6-Diphenylpyrazin-2-yl)(propan-2-yl)amino)butoxy)acetic acid (Compound 436b′). To a solution of 13c (0.21 g, 0.44 mmol) in MeOH (2 mL), was added 1N sodium hydroxide (1.0 mL). The reaction mixture was heated to reflux under stirring for a period of 1 hour and then cooled to ambient temperature. The resulting solution was concentrated in vacuo and the resulting residue dissolved in water (10 mL). The aqueous solution was then washed with Et₂O (2×10 mL). The aqueous phase was separated and acidified to pH˜2 with 1M HCl. The acidified aqueous layer was extracted with EtOAc (3×15 mL) and the combined organic extracts were dried (MgSO₄), filtered, and concentrated in vacuo to afford Compound 436b′ (0.17 g, 91%). MS (M−H): 425.0. The crude acid was used directly in the subsequent step.

### Example 6

### Synthesis of 2-(4-((5,6-Diphenylpyrazin-2-yl)(perdeutero-propan-2-yl)amino)butoxy)-N-(methylsulfonyl)acetamide (Compound 536b′)

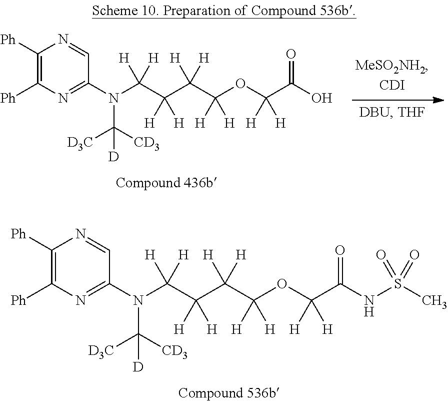 [SMILES: [2H]C([2H])([2H])C([2H])(N(CCCCOCC(=O)O)c1cnc(-c2ccccc2)c(-c2ccccc2)n1)C([2H])([2H])[2H] ; [2H]C([2H])([2H])C([2H])(N(CCCCOCC(=O)NS(C)(=O)=O)c1cnc(-c2ccccc2)c(-c2ccccc2)n1)C([2H])([2H])[2H]]

2-(4-((5,6-Diphenylpyrazin-2-yl)(perdeutero-propan-2-yl)amino)butoxy)-N-(methylsulfonyl)acetamide (Compound 536b′). To a solution of acid 436b′ (0.16 g, 0.38 mmol) in THF (3 mL), was added 1,1′-carbonyldiimidazole (CDI, 67 mg, 0.41 mmol, 1.1 equiv) and the solution was stirred at ambient temperature for 30 minutes then heated to reflux for an additional 30 minutes. After cooling the solution to ambient temperature, methanesulfonamide (36 mg, 0.38 mmol, 1.02 equiv) was added and the mixture stirred for 10 minutes at ambient temperature. To the stirred solution, was then added 1,8-diazabicyclo[5.4.0]undec-7-ene (DBU, 60 μL, 0.39 mmol, 1.03 equiv) and the mixture was stirred at ambient temperature for 12 hours then diluted with 1N HCl and extracted with Et₂O (3×20 mL). The combined organic layers were washed successively with water and brine. The resulting organic layer was dried (MgSO₄), filtered and concentrated in vacuo. The resulting residue was purified by column chromatography (SiO₂, 5-10% MeOH/CHCl₃) to afford Compound 536b′ as a pale yellow solid (0.11 g, 58%). ¹H NMR (CDCl₃, 400 MHz) δ 8.19 (s, 1H), 7.44 (m, 2H), 7.35 (m, 2H), 7.30-7.21 (m, 6H), 3.97 (s, 2H), 3.59 (t, J=6.0 Hz, 2H), 3.45 (t, J=6.8 Hz, 2H), 3.29 (s, 3H), 1.75-1.70 (m, 4H); MS (M+H): 503.9.

### Example 7

### Synthesis of 4-(Perdeutero-propan-2-yl)aminobutan-1-ol (11c)

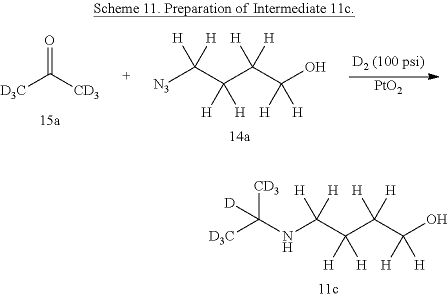

4-(perdeutero-propan-2-yl)aminobutan-1-ol (11c). Commercially available 4-azidobutan-1-ol (0.50 g, 4.34 mmol) was dissolved in acetone-d₆ (15 mL; Cambridge Isotope Laboratories, 98.8 atom % D) within a Parr bomb apparatus. To this solution was added, platinum (IV) oxide (50 mg) and the apparatus charged to 100 psi with D₂ (Medical-Technology Laboratories, Inc, 99.999 atom % D). The mixture was stirred at ambient temperature for 15 h then purged with nitrogen gas. The suspension was filtered through Celite® and the filter cake washed with EtOAc. The filtrate was concentrated in vacuo to afford d₇-aminoalcohol 11c (0.54 g, 90%); MS (M+H): 139.3.

### Example 8

### Synthesis of 1,1,2,2,3,3,4,4-d₈-4-(Perdeutero-propan-2-yl)aminobutan-1-ol (11a)

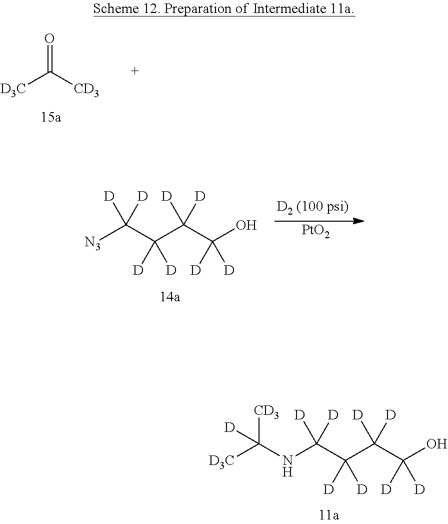

1,1,2,2,3,3,4,4-d₈-4-((5,6-Diphenylpyrazin-2-yl)(perdeutero-propan-2-yl)aminobutan-1-ol (11a). d₈-Azidoalcohol 14a (0.56 g, 4.55 mmol, prepared as described in Example 9) was dissolved in acetone-d₆ (18 mL; Cambridge Isotope Laboratories, 98.8 atom % D) within a Parr bomb apparatus. To this solution was added platinum (IV) oxide (0.11 g) and the apparatus charged to 100 psi with D₂ (Medical-Technology Laboratories, Inc, 99.999 atom % D). The mixture was stirred at ambient temperature for 15 h then purged with nitrogen gas. The suspension was filtered through Celite® and the filter cake washed with EtOAc. The filtrate was concentrated in vacuo to afford d₁₅-aminoalcohol 11a (0.63 g, 94%). MS (M+H): 147.2.

### Example 9

### Synthesis of 1,1,2,2,3,3,4,4-d₈-4-Azidobutan-1-ol (14a)

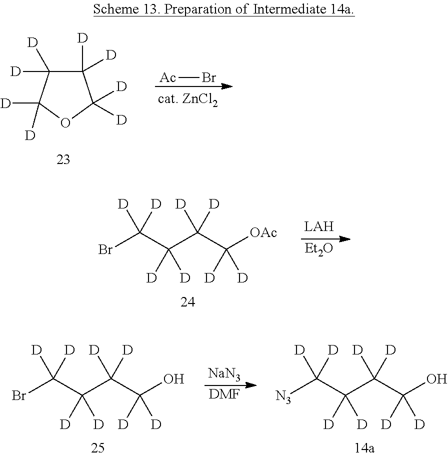

Step 1. 1,1,2,2,3,3,4,4-d₈-4-Bromobutyl acetate (24). To a suspension of ZnCl₂ (17 mg, 0.124 mmol, 0.005 equiv) in THF-d₈ (2.0 g, Cambridge Isotope Laboratories, 99.5 atom % D) at 0° C., was added dropwise acetyl bromide (2.2 mL, 29.94 mmol, 1.2 equiv). The mixture was stirred to ambient temperature over a period of 30 minutes then diluted dropwise with MeOD (2 ml) followed by aqueous NaHCO₃ (10 mL). The mixture was extracted with EtOAc (3×15 mL) and the combined organic layers were washed successively with saturated aqueous NaHCO₃ and brine. The organic layer was dried (MgSO₄), filtered and concentrated in vacuo to afford 24 (3.38 g, 67%).

Step 2. 1,1,2,2,3,3,4,4-d₈-4-Bromobutan-1-ol (25). To a suspension of lithium aluminum hydride (0.76 g, 19.97 mmol, 1.22 equiv) in Et₂O (20 mL) at 0° C., was added a solution of 24 (3.38 g, 16.64 mmol) in Et₂O (30 mL). The mixture was stirred to ambient temperature over a period of 30 minutes then diluted dropwise with aqueous saturated Na₂SO₄ until the complete formation of a cake was observed. The cake was filtered through Celite® and the filter cake washed with Et₂O. The ethereal filtrate was dried (MgSO₄), filtered and concentrated in vacuo to afford 25 (1.9 g, 71%).

Step 3. 2-(1,1,2,2,3,3,4,4-d₈-4-Azidobutan-1-ol (14a). To a solution of 25 (1.9 g, 11.8 mmol) in DMF (12 mL), was added sodium azide (1.53 g, 23.6 mmol, 2.0 equiv) and the mixture was stirred at ambient temperature for 18 hours then diluted with water (40 mL). The resulting solution was then extracted with Et₂O (3×50 mL) and the combined organic layers were washed successively with water and brine. The organic layers was then dried (MgSO₄), filtered and concentrated in vacuo to afford d₈-azidoalcohol 14a (1.2 g, 83%).

### Example 10

### Synthesis of 1,1,2,2,3,3,4,4-d₈-4-(Propan-2-yl)aminobutan-1-ol (11b)

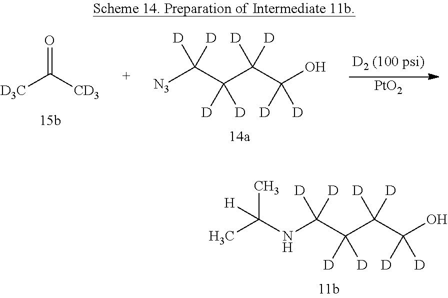

1,1,2,2,3,3,4,4-d₈-4-(Propan-2-yl)aminobutan-1-ol (11b). d₈-Azidoalcohol 14a (0.90 g, 7.30 mmol, prepared as described above) was dissolved in acetone (40 mL). To this solution was added, platinum (IV) oxide (0.11 g) and the mixture was purged with argon then placed under a H₂ atmosphere and stirred at ambient temperature for 18 hours. The suspension was filtered through Celite® and the filter cake washed with EtOAc. The filtrate was concentrated in vacuo to afford d₈-aminoalcohol 11b (0.90 g, 89%). MS (M+H): 140.2.

### Example 11

### Evaluation of Metabolic Stability in Human Liver Microsomes

Human liver microsomes (20 mg/mL) are available from Xenotech, LLC (Lenexa, Kans.). β-nicotinamide adenine dinucleotide phosphate, reduced form (NADPH), magnesium chloride (MgCl₂), and dimethyl sulfoxide (DMSO) are available from Sigma-Aldrich.

7.5 mM stock solutions of test compounds are prepared in DMSO. The 7.5 mM stock solutions are diluted to 12.5-50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 μL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 0.5 mg/mL human liver microsomes, 0.25-1.0 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold ACN with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for NS-304, MRE-269 and the positive control, 7-ethoxycoumarin (1 μM). Testing is done in triplicate.

The in vitro t_{1/2}s for test compounds are calculated from the slopes of the linear regression of % parent remaining (ln) vs incubation time relationship:

in vitro t_{1/2}=0.693/k

k=−[slope of linear regression of % parent remaining (ln) vs incubation time].

Data analysis is performed using Microsoft Excel Software.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. A compound of Formula I: 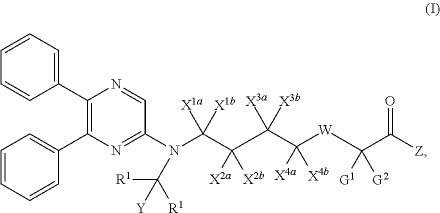
or a pharmaceutically acceptable salt thereof, wherein:
W is O, CH₂ or CD₂;
each of G¹ and G² is independently hydrogen or deuterium;
each R¹ is independently —CD₃, —CD₂H, —CDH₂, or —CH₃;
Z is —OH or —NHSO₂CH₃ or —NHSO₂CD₃;
each of X^{1a}, X^{1b}, X^{2a}, X^{2b}, X^{3a}, X^{3b}, X^{4a} and X^{4b} is independently selected from hydrogen and deuterium; and
Y is independently selected from hydrogen and deuterium;
provided that if each R¹ is —CH₃ and each of X^{1a}, X^{1b}, X^{2a}, X^{2b}, X^{3a}, X^{3b}, X^{4a} and X^{4b} is hydrogen, then Y is deuterium.

2. The compound of claim 1, wherein the compound is of Formula Ib′: 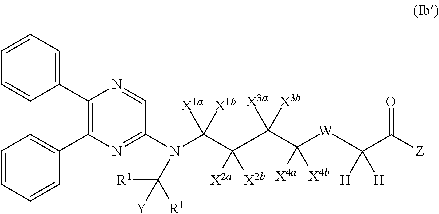
or a pharmaceutically acceptable salt thereof.

3. The compound of claim 1, wherein the compound is of Formula Ib: 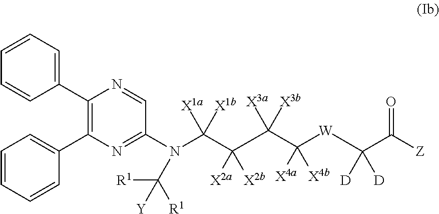
or a pharmaceutically acceptable salt thereof.

4. The compound of claim 1, wherein each R¹ is independently —CD₃ or —CH₃.

5. The compound of claim 4, wherein X^{1a}═X^{1b}; X^{2a}═X^{2b}; X^{3a}═X^{3b}; and X^{4a}═X^{4b}.

6. The compound of claim 4, wherein X^{1a}═X^{1b}; X^{2a}═X^{2b}; X^{3a}═X^{3b}; or X^{4a}═X^{4b}.

7. The compound of claim 6, wherein X^{4a}═X^{4b}.

8. (canceled)

9. (canceled)

10. The compound of claim 4, wherein Y is hydrogen.

11. The compound of claim 4, wherein Y is deuterium.

12. The compound of claim 6, wherein X^{1a}═X^{1b}.

13. (canceled)

14. (canceled)

15. The compound of claim 6, wherein X^{2a}═X^{2b}.

16. (canceled)

17. (canceled)

18. The compound of claim 6, wherein X^{3a}═X^{3b}.

19. (canceled)

20. (canceled)

21. The compound of claim 5, wherein every X is deuterium.

22. The compound of claim 5, wherein every X is hydrogen.

23-33. (canceled)

34. A compound of claim 1, wherein Z is —OH and the compound is selected from the following:

| Com- | | | | | | | | | | |
|---|---|---|---|---|---|---|---|---|---|---|
| pound | X^{1a} | X^{1b} | X^{2a} | X^{2b} | X^{3a} | X^{3b} | X^{4a} | X^{4b} | R¹ | Y |
| 100 | D | D | D | D | D | D | D | D | CD₃ | D |
| 101 | D | D | D | D | D | D | D | D | CD₃ | H |
| 102 | D | D | D | D | D | D | D | D | CH₃ | D |
| 103 | D | D | D | D | D | D | D | D | CH₃ | H |
| 104 | H | H | D | D | D | D | H | H | CD₃ | D |
| 105 | H | H | D | D | D | D | H | H | CD₃ | H |
| 106 | H | H | D | D | D | D | H | H | CH₃ | D |
| 107 | H | H | D | D | D | D | H | H | CH₃ | H |
| 108 | D | D | H | H | H | H | D | D | CD₃ | D |
| 109 | D | D | H | H | H | H | D | D | CD₃ | H |
| 110 | D | D | H | H | H | H | D | D | CH₃ | D |
| 111 | D | D | H | H | H | H | D | D | CH₃ | H |
| 112 | D | D | D | D | H | H | H | H | CD₃ | D |
| 113 | D | D | D | D | H | H | H | H | CD₃ | H |
| 114 | D | D | D | D | H | H | H | H | CH₃ | D |
| 115 | D | D | D | D | H | H | H | H | CH₃ | H |
| 116 | H | H | H | H | D | D | D | D | CD₃ | D |
| 117 | H | H | H | H | D | D | D | D | CD₃ | H |
| 118 | H | H | H | H | D | D | D | D | CH₃ | D |
| 119 | H | H | H | H | D | D | D | D | CH₃ | H |
| 120 | D | D | H | H | D | D | H | H | CD₃ | D |
| 121 | D | D | H | H | D | D | H | H | CD₃ | H |
| 122 | D | D | H | H | D | D | H | H | CH₃ | D |
| 123 | D | D | H | H | D | D | H | H | CH₃ | H |
| 124 | H | H | D | D | H | H | D | D | CD₃ | D |
| 125 | H | H | D | D | H | H | D | D | CD₃ | H |
| 126 | H | H | D | D | H | H | D | D | CH₃ | D |
| 127 | H | H | D | D | H | H | D | D | CH₃ | H |
| 128 | D | D | H | H | H | H | H | H | CD₃ | D |
| 129 | D | D | H | H | H | H | H | H | CD₃ | H |
| 130 | D | D | H | H | H | H | H | H | CH₃ | D |
| 131 | D | D | H | H | H | H | H | H | CH₃ | H |
| 132 | H | H | H | H | H | H | D | D | CD₃ | D |
| 133 | H | H | H | H | H | H | D | D | CD₃ | H |
| 134 | H | H | H | H | H | H | D | D | CH₃ | D |
| 135 | H | H | H | H | H | H | D | D | CH₃ | H |
| 136 | H | H | H | H | H | H | H | H | CD₃ | D |
| 137 | H | H | H | H | H | H | H | H | CD₃ | H |
| 138 | H | H | H | H | H | H | H | H | CH₃ | D |

or a pharmaceutically acceptable salt of any of the foregoing compounds.

35. A compound of claim 1, wherein Z is —NHSO₂CH₃ and the compound is selected from

| Com- | | | | | | | | | | |
|---|---|---|---|---|---|---|---|---|---|---|
| pound | X^{1a} | X^{1b} | X^{2a} | X^{2b} | X^{3a} | X^{3b} | X^{4a} | X^{4b} | R¹ | Y |
| 200 | D | D | D | D | D | D | D | D | CD₃ | D |
| 201 | D | D | D | D | D | D | D | D | CD₃ | H |
| 202 | D | D | D | D | D | D | D | D | CH₃ | D |
| 203 | D | D | D | D | D | D | D | D | CH₃ | H |
| 204 | H | H | D | D | D | D | H | H | CD₃ | D |
| 205 | H | H | D | D | D | D | H | H | CD₃ | H |
| 206 | H | H | D | D | D | D | H | H | CH₃ | D |
| 207 | H | H | D | D | D | D | H | H | CH₃ | H |
| 208 | D | D | H | H | H | H | D | D | CD₃ | D |
| 209 | D | D | H | H | H | H | D | D | CD₃ | H |
| 210 | D | D | H | H | H | H | D | D | CH₃ | D |
| 211 | D | D | H | H | H | H | D | D | CH₃ | H |
| 212 | D | D | D | D | H | H | H | H | CD₃ | D |
| 213 | D | D | D | D | H | H | H | H | CD₃ | H |
| 214 | D | D | D | D | H | H | H | H | CH₃ | D |
| 215 | D | D | D | D | H | H | H | H | CH₃ | H |
| 216 | H | H | H | H | D | D | D | D | CD₃ | D |
| 217 | H | H | H | H | D | D | D | D | CD₃ | H |
| 218 | H | H | H | H | D | D | D | D | CH₃ | D |
| 219 | H | H | H | H | D | D | D | D | CH₃ | H |
| 220 | D | D | H | H | D | D | H | H | CD₃ | D |
| 221 | D | D | H | H | D | D | H | H | CD₃ | H |
| 222 | D | D | H | H | D | D | H | H | CH₃ | D |
| 223 | D | D | H | H | D | D | H | H | CH₃ | H |
| 224 | H | H | D | D | H | H | D | D | CD₃ | D |
| 225 | H | H | D | D | H | H | D | D | CD₃ | H |
| 226 | H | H | D | D | H | H | D | D | CH₃ | D |
| 227 | H | H | D | D | H | H | D | D | CH₃ | H |
| 228 | D | D | H | H | H | H | H | H | CD₃ | D |
| 229 | D | D | H | H | H | H | H | H | CD₃ | H |
| 230 | D | D | H | H | H | H | H | H | CH₃ | D |
| 231 | D | D | H | H | H | H | H | H | CH₃ | H |
| 232 | H | H | H | H | H | H | D | D | CD₃ | D |
| 233 | H | H | H | H | H | H | D | D | CD₃ | H |
| 234 | H | H | H | H | H | H | D | D | CH₃ | D |
| 235 | H | H | H | H | H | H | D | D | CH₃ | H |
| 236 | H | H | H | H | H | H | H | H | CD₃ | D |
| 237 | H | H | H | H | H | H | H | H | CD₃ | H |
| 238 | H | H | H | H | H | H | H | H | CH₃ | D |

or a pharmaceutically acceptable salt of any of the foregoing compounds.

36. A compound of claim 1, wherein Z is —NHSO₂CD₃ and the compound is selected from

| Com- | | | | | | | | | | |
|---|---|---|---|---|---|---|---|---|---|---|
| pound | X^{1a} | X^{1b} | X^{2a} | X^{2b} | X^{3a} | X^{3b} | X^{4a} | X^{4b} | R¹ | Y |
| 300 | D | D | D | D | D | D | D | D | CD₃ | D |
| 301 | D | D | D | D | D | D | D | D | CD₃ | H |
| 302 | D | D | D | D | D | D | D | D | CH₃ | D |
| 303 | D | D | D | D | D | D | D | D | CH₃ | H |
| 304 | H | H | D | D | D | D | H | H | CD₃ | D |
| 305 | H | H | D | D | D | D | H | H | CD₃ | H |
| 306 | H | H | D | D | D | D | H | H | CH₃ | D |
| 307 | H | H | D | D | D | D | H | H | CH₃ | H |
| 308 | D | D | H | H | H | H | D | D | CD₃ | D |
| 309 | D | D | H | H | H | H | D | D | CD₃ | H |
| 310 | D | D | H | H | H | H | D | D | CH₃ | D |
| 311 | D | D | H | H | H | H | D | D | CH₃ | H |
| 312 | D | D | D | D | H | H | H | H | CD₃ | D |
| 313 | D | D | D | D | H | H | H | H | CD₃ | H |
| 314 | D | D | D | D | H | H | H | H | CH₃ | D |
| 315 | D | D | D | D | H | H | H | H | CH₃ | H |
| 316 | H | H | H | H | D | D | D | D | CD₃ | D |
| 317 | H | H | H | H | D | D | D | D | CD₃ | H |
| 318 | H | H | H | H | D | D | D | D | CH₃ | D |
| 319 | H | H | H | H | D | D | D | D | CH₃ | H |
| 320 | D | D | H | H | D | D | H | H | CD₃ | D |
| 321 | D | D | H | H | D | D | H | H | CD₃ | H |
| 322 | D | D | H | H | D | D | H | H | CH₃ | D |
| 323 | D | D | H | H | D | D | H | H | CH₃ | H |
| 324 | H | H | D | D | H | H | D | D | CD₃ | D |
| 325 | H | H | D | D | H | H | D | D | CD₃ | H |
| 326 | H | H | D | D | H | H | D | D | CH₃ | D |
| 327 | H | H | D | D | H | H | D | D | CH₃ | H |
| 328 | D | D | H | H | H | H | H | H | CD₃ | D |
| 329 | D | D | H | H | H | H | H | H | CD₃ | H |
| 330 | D | D | H | H | H | H | H | H | CH₃ | D |
| 331 | D | D | H | H | H | H | H | H | CH₃ | H |
| 332 | H | H | H | H | H | H | D | D | CD₃ | D |
| 333 | H | H | H | H | H | H | D | D | CD₃ | H |
| 334 | H | H | H | H | H | H | D | D | CH₃ | D |
| 335 | H | H | H | H | H | H | D | D | CH₃ | H |
| 336 | H | H | H | H | H | H | H | H | CD₃ | D |
| 337 | H | H | H | H | H | H | H | H | CD₃ | H |
| 338 | H | H | H | H | H | H | H | H | CH₃ | D |

or a pharmaceutically acceptable salt of any of the foregoing compounds.

37. The compound of claim 1, wherein any atom not designated as deuterium in any of the embodiments set forth above is present at its natural isotopic abundance.

38. A pyrogen-free composition comprising an effective amount of a compound of claim 1 or a pharmaceutically acceptable salt of said compound; and a carrier.

39. The composition of claim 38, wherein the composition is formulated for pharmaceutical use and the carrier is a pharmaceutically acceptable carrier.

40. (canceled)

41. A method of treating a patient suffering from, or susceptible to, a disease or condition that may be treated by inhibition of platelet aggregation, vasodilation, inhibition of lipid deposition, inhibition of leukocyte activation, or a combination thereof, comprising the step of administering to the patient in need thereof an effective amount of a compound of claim 1.

42. A method of treating a disease or condition selected from pulmonary arterial hypertension, peripheral vascular diseases, systemic lupus erythematosus, reocclusion or restenosis after percutaneous transluminal coronary angioplasty (PTCA), arteriosclerosis, thrombosis, diabetic neuropathy, diabetic nephropathy, hypertension, ischemic diseases, transient ischemic attack, and glomerulonephritis, comprising the step of administering to the patient in need thereof an effective amount of a composition of a compound of claim 1.

43-44. (canceled)
